(19) United States
(12) Patent Application Publication
(10) Pub. No.: US 2026/0102355 A1
(43) Pub. Date: Apr. 16, 2026

(54) ORAL PREPARATION COMPRISING CHICORY DIETARY FIBER OR CASSAVA STARCH

(71) Applicant: LG H&H CO., LTD., Seoul (KR)

(72) Inventors: Nojin PARK, Seoul (KR); Jooyeon RYOO, Seoul (KR); Seung-Hyun JUN, Seoul (KR); Won Kyung LEE, Seoul (KR); Wonkyung CHOI, Seoul (KR); Nae Gyu KANG, Seoul (KR)

(73) Assignee: LG H&H CO., LTD., Seoul (KR)

(21) Appl. No.: 19/113,030

(22) PCT Filed: Nov. 30, 2023
(86) PCT No.: PCT/KR2023/019499

§ 371 Date: Mar. 19, 2025

(30) Foreign Application Priority Data
Jan. 4, 2023 (KR) 10-2023-0001293

(51) Int. Cl. A61K 9/20; A61K 8/02; A61K 8/25; A61K 8/73; A61K 8/9789; A61K 33/30; A61K 33/34; A61P 1/02; A61Q 11/00

(52) U.S. Cl. CPC A61K 9/2086; A61K 8/0233; A61K 8/25; A61K 8/732; A61K 8/9789; A61K 9/2009; A61K 9/2013; A61K 9/2054; A61K 9/2059; A61K 9/2068; A61K 33/30; A61K 33/34; A61P 1/02; A61Q 11/00

(57) ABSTRACT

According to one embodiment of the present invention, the present invention provides an oral preparation comprising: an adhesive layer comprising an adhesive polymer; and a functional layer that provides one or more functions selected from halitosis suppression, promotion of saliva secretion, and antibacterial effects, wherein the adhesive polymer comprises one or more selected from the group consisting of cassava starch in which linamarin is removed and chicory dietary fiber.

### TECHNICAL FIELD

The present invention relates to an oral mucosal preparation exhibiting low irritation and excellent adhesion to oral mucosa, comprising an adhesive layer comprising chicory dietary fiber or cassava starch in which linamarin is removed, and a functional layer containing functional ingredients for removal or alleviation of halitosis.

### BACKGROUND ART

Halitosis refers to an unpleasant odor that occurs in the mouth. Although halitosis does not cause pain to the patient, it may occur during breathing, conversation, or chewing, potentially impacting interpersonal relationships negatively. Halitosis contributes to psychological distress and hinders the normal social life of patients, causing them to experience significant discomfort. Halitosis resulting from disorders in the digestive system, such as the stomach or intestines, is estimated to account for less than 10% of all cases, whereas approximately 90% of halitosis are attributed to gram-negative anaerobic bacteria, which cause the breakdown of proteins in food debris remaining in the mouth, oral cells, saliva, and the like, resulting in the production of volatile sulfur compounds (VSCs) such as hydrogen sulfide (H₂S), methyl mercaptan (CH₃SH), and dimethyl sulfide ((CH₃)₂S).

Conditions that promote the occurrence of halitosis include poor oral hygiene due to food debris remaining between teeth or on dental appliances such as dentures, as well as inflammatory oral diseases such as periodontitis and tonsillitis. In addition, the proliferation of halitosis-causing bacteria in the oral cavity, attributed to factors that reduce saliva secretion—such as smoking, sleep, fasting, and dry mouth—is known to cause halitosis. Accordingly, there are known measures to improve halitosis, such as consuming saliva secretion-promoting agents, neutralizing VSCs, and suppressing the activity of halitosis-causing bacteria, in addition to eliminating the factors that decrease saliva secretion, such as smoking.

Xylitol, known as a sugar substitute, has been reported to inhibit acid production and energy acquisition of Streptococcus mutans (S. mutans), thereby not only reducing the population of S. mutans but also promoting saliva secretion. In addition to zinc ions (Zn²⁺) and copper ions (Cu²⁺), several metal ions, are known to inhibit the formation of VSCs due to their strong binding affinity to sulfides such as S²⁻ and CH₃S⁻. Moreover, some metal ions such as zinc ions (Zn²⁺) are known to exhibit not only a VSC-neutralizing effect but also an antimicrobial effect against halitosis-causing bacteria, including Porphyromonas gingivalis (P. gingivalis), Fusobacterium nucleatum (F. nucleatum), Tannerella forsythia (T. forsythia), Prevotella intermedia (P. intermedia), and Prevotella nigrescens (P. nigrescens). Accordingly, products for reducing halitosis, comprising these ingredients, are known. Further, it has also been reported that the effectiveness is enhanced when active ingredients for antibacterial activity and saliva stimulation are continuously released in the oral cavity.

As a result, various products in forms such as gums and candies comprising active ingredients for oral health improvement, have been developed to promote the sustained release of active ingredients in the oral cavity during consumption. However, the duration of sustained release is known to range from 5 minutes to a maximum of 15 minutes, which is insufficient. As an alternative, preparations comprising adhesive ingredients such as carboxymethyl cellulose (CMC), Carbopol (polyacrylic acid), and polyvinyl pyrrolidone (PVP) have been developed, which are designed to adhere to specific locations in the oral cavity, enabling sustained use compared to conventional products, and are available in the form of patches or tablets. However, CMC, Carbopol, or PVP are synthetic adhesive polymers known to cause irritation to the oral mucosa through ionic interaction, which can lead to mucosal pain. Another known alternative is gum arabic, a natural adhesive polymer; however, this natural polymer still contains carboxyl groups (COOH groups), which pose the issue of ionic interactions with the oral mucosa.

Moreover, when a fluid film forms on the mucosa under conditions of high saliva secretion, the interaction between the adhesive polymer and the mucosa is reduced, making adhesion difficult. Therefore, there is a demand for preparations that employ polymers without ionic interactions and feature a highly adhesive layer with strong adherence to the oral mucosa, even under conditions of high saliva secretion.

In light of this background, the present inventors propose an adhesive preparation comprising chicory dietary fiber or cassava starch (tapioca) in addition to silica, and a bilayer preparation comprising active ingredients for the elimination or alleviation of halitosis as well as an adhesive layer, in order to address conventional issues. That is, the present invention aims to provide an improved preparation that uses chicory dietary fiber or cassava starch as an adhesive polymer, which can exhibit adhesion without containing carboxyl groups (—COOH) and undergoing ionic interactions with oral mucosa, thereby exhibiting similar or superior adhesiveness compared to that of conventionally known adhesive polymers and reduced irritation to the oral mucosa caused by sustained use of adhesive agents for suppression or alleviation of halitosis. Additionally, when silica is used as an excipient in the adhesive layer of the bilayer tablet, the tablet may exhibit superior adhesive strength to the oral mucosa compared to other excipients. Accordingly, the present invention also aims to provide a tableted preparation with enhanced strength.

### DISCLOSURE

### Technical Problem

An object of the present invention is to provide an oral preparation comprising: an adhesive layer comprising one or more selected from the group consisting of cassava starch in which linamarin is removed and chicory dietary fiber; and a functional layer that provides one or more functions selected from halitosis suppression, promotion of saliva secretion, and antimicrobial effects, wherein the oral preparation exhibits excellent adhesion and low oral mucosal irritation.

Another object of the present invention is to provide an oral adhesive composition using a polymer comprising one or more selected from the group consisting of cassava starch in which linamarin is removed and chicory dietary fiber as an oral adhesive.

### Technical Solution

The present disclosure will be described in detail as follows. Meanwhile, each description and embodiment described herein may be applied to other descriptions and embodiments, respectively. That is, all combinations of various elements described herein fall within the scope of the present invention. Further, the scope of the present invention is not limited by the specific description described below.

One embodiment of the present invention provides an oral preparation comprising: an adhesive layer comprising an adhesive polymer; and a functional layer, wherein the adhesive polymer comprises one or more selected from the group consisting of cassava starch in which linamarin is removed and chicory dietary fiber.

Another embodiment of the present invention provides an oral preparation in which the functional layer provides one or more functions selected from suppressing halitosis, promoting saliva secretion, reducing tooth plaque, reducing gingivitis, reducing periodontitis, alleviating periodontal disease, and alleviating dental caries.

Still another embodiment of the present invention provides an oral preparation wherein the oral preparation is in the form of a bilayer tablet.

Still another embodiment of the present invention provides an oral preparation in which the adhesive layer further comprises silica as an excipient and wherein the oral preparation is in any one formulation selected from the group consisting of halitosis removing agents, dry mouth relievers, solid oral cleansers, solid toothpastes, and sustained-release candies.

Still another embodiment of the present invention provides an oral preparation in which the excipient is included in an amount of 20 wt % to 40 wt % based on the total weight of raw materials of the adhesive layer.

Still another embodiment of the present invention provides an oral preparation in which the silica has a particle diameter of 1 nm or more and 50 μm or less.

Still another embodiment of the present invention provides an oral preparation in which the functional layer comprises, as a halitosis suppressant, one or more selected from the group consisting of salts of copper ions (Cu²⁺), zinc ions (Zn²⁺), and tin ions (Sn²⁺).

Still another embodiment of the present invention provides an oral preparation in which the functional layer comprises the halitosis suppressant in an amount of 0.1 wt % to 5 wt % based on the total weight of the functional layer.

Still another embodiment of the present invention provides an oral preparation in which the functional layer comprises a saliva stimulant in an amount of 50 wt % to 80 wt % based on the total weight of the functional layer.

Still another embodiment of the present invention provides an oral preparation in which the adhesive layer is included in an amount of 2 wt % to 50 wt % based on the total weight of the oral preparation.

Still another embodiment of the present invention provides an oral preparation in which the functional layer is included in an amount of 20 wt % to 85 wt % based on the total weight of the oral preparation.

Still another embodiment of the present invention provides an oral preparation wherein the oral preparation exhibits a strength of 1000 gf or more and less than 4000 gf.

Still another embodiment of the present invention provides an oral preparation wherein the oral preparation further comprises a layer that performs one or more functions selected from the group consisting of surface coating, dissolution rate control, strength control, tooth surface polishing, whitening, and pH adjustment.

Still another embodiment of the present invention provides an oral adhesive composition comprising a polymer, wherein the polymer comprises one or more selected from the group consisting of cassava starch in which linamarin is removed and chicory dietary fiber and functions as an oral adhesive.

### Advantageous Effects

The oral adhesive composition of the present invention, comprising one or more selected from the group consisting of cassava starch in which linamarin is removed and chicory dietary fiber, exhibits reduced irritation to the oral mucosa and excellent adhesiveness.

Additionally, an oral preparation comprising the oral adhesive composition exhibits low oral mucosal irritation and demonstrates high adhesion to the oral mucosa even in situations with high saliva secretion, thereby offering excellent applicability.

### BRIEF DESCRIPTION OF THE DRAWINGS

FIG. 1 is an image of an oral preparation according to an embodiment of the present invention.

FIG. 2 is a schematic diagram illustrating a method for measuring the hydrophilicity of silica included in an oral preparation according to one embodiment of the present invention.

FIG. 3 is an image of a test process for measuring the adhesive strength of an oral preparation according to an embodiment of the present invention.

FIG. 4 is an image of a test process for measuring the strength of an oral preparation according to an embodiment of the present invention.

FIG. 5 is a graph comparing the adhesive strength of an oral preparation according to an embodiment of the present invention.

FIG. 6 is a graph comparing the VSC neutralization rate according to the presence or absence of a halitosis suppressant in an oral preparation according to one embodiment of the present invention.

FIG. 7 is a graph comparing the dissolution rate of an oral preparation according to an embodiment of the present invention.

### MODE FOR CARRYING OUT THE INVENTION

The present disclosure will be described in detail by way of Examples. The Examples are provided to specifically explain the present invention, and the scope of the present invention is not limited by the Examples.

The present invention may undergo various modifications and take on multiple forms, and specific embodiments are illustrated in the drawings and described in detail in the specification. However, the present invention is not limited to the specific embodiments described herein and should be understood to encompass all modifications, equivalents, and alternative embodiments within the spirit and technical scope of the invention of the present invention.

In the description of each figure, similar reference numerals are used for similar components. In the attached figures, the dimensions of the structures are enlarged for the clarity of the present invention. Terms such as “first” and “second” may be used to describe various components, but these components should not be limited by the aforementioned terms. The aforementioned terms are used solely for the purpose of distinguishing one component from another component. For example, without departing from the scope of the present invention, the first component may be referred to as the second component, and similarly, the second component may also be referred to as the first component. Singular expressions include plural expressions unless the context clearly dictates otherwise.

In the present invention, terms such as “including”, “comprising”, or “having” are intended to indicate the presence of features, numbers, steps, operations, components, parts, or combinations thereof as disclosed in the specification, but do not preclude the presence or addition of one or more other features, numbers, steps, operations, components, parts, or combinations thereof. Additionally, when a component such as a layer, film, region, or plate is described as being “on” another part, this includes not only cases where the part is “directly above” the other, but also cases where another part is present in between.

Additionally, in this specification, when a part such as a layer, film, region, or plate is described as being formed on another part, the formation direction is not limited to the upper direction but also includes lateral or lower directions. Conversely, when a part such as a layer, film, region, or plate is described as being “under” another part, this includes not only cases where the part is “directly below” the other, but also cases where another part is present in between.

One embodiment of the present invention provides an oral preparation comprising: an adhesive layer comprising an adhesive polymer; and a functional layer, wherein the adhesive polymer comprises one or more selected from the group consisting of cassava starch in which linamarin is removed and chicory dietary fiber.

Another embodiment of the present invention provides an oral preparation in which the functional layer provides one or more functions selected from suppressing halitosis, promoting saliva secretion, reducing tooth plaque, reducing gingivitis, reducing periodontitis, alleviating periodontal disease, and alleviating dental caries.

In the case of carboxy methyl cellulose (CMC), Carbopol (polyacrylic acid), and polyvinyl pyrrolidone (PVP), which are known as adhesive components used in conventional technology, these adhesive synthetic polymers interact ionically with the oral mucosa, causing mucosal pain. Another alternative, the natural adhesive polymer gum arabic, also contains carboxyl groups (—COOH), and thus cannot resolve the issue of ionic interactions with the oral mucosa. In contrast, chicory dietary fiber and cassava starch, from which linamarin is removes, a natural adhesive polymer, both of which are included in an oral preparation according to an embodiment of the present invention, do not contain carboxyl groups (—COOH) or interact ionically with the oral mucosa. Further, they have adhesive strength similar to or superior to that of known adhesive polymers and may reduce irritation to the oral mucosa caused by the sustained use of an adhesive preparation for suppressing or alleviating halitosis.

Additionally, the oral preparation according to one embodiment of the present invention can control the dissolution rate. The oral preparation may be provided in the form of effervescent tablets using sodium bicarbonate (NaHCO₃) and citric acid and may further comprise hydrophilic ingredients such as salt or sugar. Additionally, the binder content may be increased to reduce the dissolution rate, and the oral preparation according to an embodiment of the present invention may be used for a duration of 1 hour to a maximum of 4 hours.

According to one embodiment of the present invention, the oral preparation may be in the form of a bilayer tablet.

FIG. 1 is an image of an oral preparation according to an embodiment of the present invention.

Referring to FIG. 1, the oral preparation comprises an adhesive layer containing an adhesive polymer and a functional layer that provides functions such as halitosis suppression, saliva secretion promotion, and antimicrobial effects. The oral preparation may be composed of a bilayer, as show in in FIG. 1, but is not limited thereto.

According to an embodiment of the present invention, the diameter of the oral preparation may be 5 mm or more and 16 mm or less, preferably 7 mm or more and 14 mm or less, and even more preferably 9 mm or more and 12 mm or less. The thickness of the oral preparation may be 2 mm or more and 6 mm or less, preferably 2.5 mm or more and 5 mm or less, and more preferably, 3 mm or more and 4 mm or less. If the diameter is 5 mm or less, the small size may cause discomfort during use, and if it is 16 mm or more, a significant portion may extend beyond the gums, making adhesion difficult. Additionally, if the thickness is 2 mm or less, the low strength may cause breakage and make tableting difficult, whereas if it is 6 mm or more, it may be too thick, leading to discomfort during use.

The raw materials of the adhesive layer may be included in an amount of 0.5% or more and 60% or less, preferably 1% or more and 55% or less, and even more preferably 2% or more and 50% or less, based on the total weight of the bilayer. The raw materials of the adhesive layer may comprise adhesive polymers, excipients, binders, lubricants, etc.

As an “adhesive polymer”, chicory dietary fiber or cassava starch (tapioca) in which linamarin is removed may be employed.

Chicory dietary fiber refers to a substance extracted from chicory plants (e.g., roots) through methods such as enzyme treatment or hydrothermal treatment. It is a polysaccharide that is not broken down by human digestive enzymes and includes inulin, with a degree of polymerization of about 2 to about 60, and oligo-fructose. Representative raw materials include Orafti GR from Beneo (Germany).

Cassava starch refers to starch extracted from cassava roots, and is a white powder rich in amylopectin which linamarin has been removed. Representative raw materials include Tapioca starch from Sanguan Wongse Industries (Thailand). Linamarin refers to a cyanogenic glucoside found in leaves and roots of plants such as cassava, lima beans, and flax. It is a glycoside compound containing cyanide residues and is considered a type of plant toxin. By itself, the compound is stable and harmless to the human body, however, upon hydrolysis, cyanide is generated therefrom, exhibiting toxicity. It is mostly found in parts that are typically not consumed but is occasionally present in edible parts. However, in the case of cassava, a significant amount of linamarin is found in the roots and leaves. Accordingly, in the present invention, linamarin was removed from cassava starch to eliminate the risk of dietary exposure to Iinamarin.

The adhesive polymer may be included in an amount of 0.5% or more and 80% or less, preferably 10% or more and 70% or less, and even more preferably 20% or more and 60% or less, based on the total weight of the raw materials of the adhesive layer. As a binder for controlling the adhesiveness of the adhesive polymer and enhancing the binding strength of the adhesive layer, one or more of gelatin, alginate, pectin, starch, hydroxy methyl cellulose, hydroxy ethyl cellulose, hydroxypropyl methylcellulose, polyvinyl acid, carboxymethyl cellulose, and the like may be included. These binders may be incorporated within the adhesive layer in an amount excluding the content of the adhesive polymer, excipients, pH adjusters, and lubricants from the total weight of the raw materials of the adhesive layer.

According to one embodiment of the present invention, the adhesive layer further comprises silica as an excipient, and the oral preparation may be in any one formulation selected from the group consisting of halitosis removing agents, dry mouth relievers, solid oral cleansers, solid toothpastes, and sustained-release candies. As used herein, the term “excipient” may typically include calcium carbonate (CaCO₃), calcium sulfate (CaSO₄), calcium phosphate (CaPO₄), starch, and the like, which are commonly used in tablets. However, silica may also be included to enhance the strength of the bilayer preparation and the adhesive strength of the adhesive layer under conditions of high saliva production. As used herein, the term “silica” refers to silica particles with a hydrophilic surface, including precipitated silica, fumed silica, colloidal silica, and the like, in terms of the manufacturing method.

Further, the excipient may be included in an amount of 1 wt % or more and 60 wt % or less, preferably 10 wt % or more and 50 wt % or less, and even more preferably 20 wt % or more and 40 wt % or less, based on the total weight of the raw materials of adhesive layer. When the excipient is included in an amount of 1 wt % or less based on the total weight of the raw materials of the adhesive layer, the effect of enhancing strength and improving adhesiveness under conditions of high saliva production may be insignificant. Conversely, if it is included in an amount of more than 50 wt %, excessive dust generation may make it difficult to mix during the tableting process.

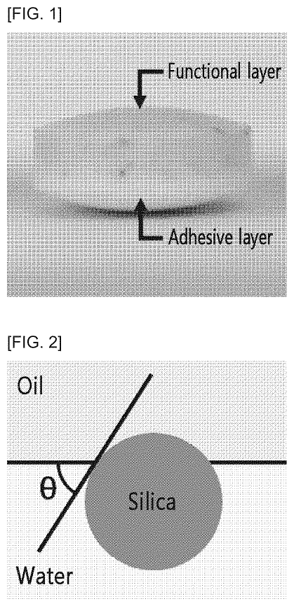

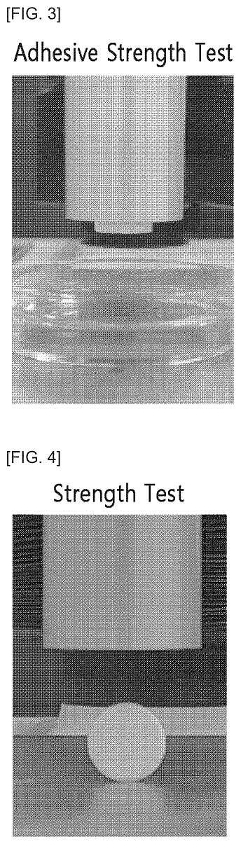

FIG. 2 is a schematic diagram illustrating a method for measuring the hydrophilicity of silica included in an oral preparation according to one embodiment of the present invention. As used herein, the term ‘hydrophilicity’ refers to a material in which the angle formed between a tangent line drawn at the point where silica particles located at the horizontal interface of water and oil (a solvent that does not mix with water) meet the interface and water at the interface is 0 degrees or more and 90 degrees or less, preferably 5 degrees or more and 85 degrees or less, and more preferably 10 degrees or more and 80 degrees or less.

Additionally, the diameter of the silica particles is 1 nm or more and 50 μm or less, preferably 1.5 nm or more and 40 μm or less, and more preferably 2 nm or more and 30 μm or less. If the diameter of the silica particles is less than 1 nm, handling becomes difficult due to dust generation during manufacturing, and the binding strength is weakened. Conversely, if the diameter is 50 μm or more, the effects of strength and adhesion enhancement are reduced.

A “lubricant” may be added to facilitate the compression and tablet ejection during manufacturing. Raw materials of the lubricant may include calcium stearate, magnesium stearate, stearic acid, sodium stearyl fumarate, talc, and the like. They may be included in an amount of 0.1% or more and 10% or less, preferably 0.3% or more and 7% or less, and more preferably 0.5% or more and 5% or less, based on the total weight of the raw materials of the adhesive layer. If the lubricant content exceeds 10% of the total weight of the raw materials of the adhesive layer, the adhesiveness may decrease. Conversely, if it is less than 0.1%, difficulties may arise during tableting due to friction.

The raw materials of the functional layer may be included in an amount of 10% or more and 95% or less, preferably 15% or more and 90% or less, more preferably 20% or more and 85% or less, and even more preferably 50% or more and 70% or less, based on the total weight of the bilayer. The raw materials of the functional layer may include halitosis suppressants, salivary secretion promoting agents, excipients, binders, lubricants, and the like, but is not limited thereto. If the weight of the functional layer exceeds 90% of the total weight of the bilayer, the adhesion may be reduced. If it is less than 10%, the effectiveness may be insignificant.

The functional layer may consist of multiple layers rather than a single layer. Additionally, when the functional layer is composed of multiple layers, each layer can perform different functions, and the layers performing the same function may be separated.

According to one embodiment of the present invention, the functional layer comprises, as a halitosis suppressant, one or more selected from the group consisting of salts of copper ions (Cu²⁺), zinc ions (Zn²⁺), and tin ions (Sn²⁺).

As used herein, the term “halitosis suppressant” refers to an ingredient that has a VSC-neutralizing effect or an antimicrobial effect against VSC-producing bacteria. As used herein, the term “VSC-neutralizing effect” refers to the inhibition of VSC volatilization due to strong ionic binding affinity to sulfides such as S²⁻ and CH₃S⁻. As used herein, the term “VSC-producing bacteria” refers to bacteria that generate VSCs, including P. gingivalis, F. nucleatum, T. forsythus, P. intermedia, and P. nigrescens.

The halitosis suppressant may contain one or more salts comprising copper ions (Cu²⁺), zinc ions (Zn²⁺), tin ions (Sn²⁺), and the like. The halitosis suppressant is included in an amount of 0.01% or more and 10% or less, preferably 0.05% or more and 7% or less, and more preferably 0.1% or more and 5% or less, based on the raw materials of the functional layer. It is preferable that the halitosis suppressant is included within the daily recommended intake.

When copper ions (Cu²⁺), zinc ions (Zn²⁺), or tin ions (Sn²⁺), which serve as halitosis suppressants, are included, they may inhibit VSC formation due to their strong binding affinity to sulfides such as S²⁻ and CH₃S⁻. Additionally, oral preparations comprising zinc ions (Zn²⁺) as the halitosis suppressant may exhibit antimicrobial effects.

The oral preparation manufactured by comprising the halitosis suppressant may continuously release the active ingredient within the oral cavity, thereby enhancing its effectiveness.

The salt of copper ion (Cu²⁺) may include one or more selected from the group consisting of copper acetylmethionate, copper acetyl tyrosinate methylsilanol, copper ascorbyl phosphate succinoyl tripeptide-34, copper aspartate, copper carbonate hydroxide, copper chlorophyll, copper chloride, copper citrate, copper DNA, copper gluconate, copper heptapeptide-14 pantothenate, copper lysinate/prolinate, copper palmitoyl heptapeptide-14, copper PCA, copper PCA methylsilanol, copper picolinate, copper sulfate, copper tetrapeptide-52 dimer, copper tripeptide-1, copper tripeptide-34 HCl, copper tripeptide-1 palmitamide, disodium EDTA-copper, and the like.

The salt of zinc ion (Zn²⁺) may include one or more selected from the group consisting of zinc acetate, zinc adenosine triphosphate, zinc adenosine triphosphate hydroxide, zinc ascorbate, zinc ascorbate hydroxide, zinc aspartate, zinc borate, zinc carbonate, zinc carbonate hydroxide, zinc carboxydecyl trisiloxane, zinc chloride, zinc chloride hydroxide, zinc citrate, zinc coceth sulfate, zinc coco-sulfate, zinc cysteinate, zinc dibutyldithiocarbamate, zinc dimethicone PEG-25 phthalate, zinc dimethicone PEG-8 succinate, zinc DNA, zinc formaldehyde sulfoxylate, zinc glucoheptonate, zinc gluconate, zinc glutamate, zinc glycinate, zinc glycinate salicylate, zinc glycyrrhetinate, zinc hydrolyzed collagen, zinc hydrolyzed hyaluronate, zinc indoleacetate hydroxide, zinc lactate, zinc laurate, zinc magnesium aspartate, zinc myristate, zinc neodecanoate, zinc nitrate, zinc oxide, zinc palmitate, zinc palmitoyl nonapeptide-14, zinc PCA, zinc pentadecene tricarboxylate, zinc peroxide, zinc phenolsulfonate, zinc picolinate, zinc pyrithione, zinc ricinoleate, zinc rosinate, zinc salicylate, zinc stearate, zinc undecylenate, zinc undecylenoyl hydrolyzed wheat protein, and the like.

The salt of tin ion (Sn²⁺) may include tin oxide or the like.

Additionally, in addition to the above ingredients, the functional layer may further include antimicrobial ingredients such as chlorhexidine, cetylpyridinium chloride, triclosan, essential oils, chlorine dioxide, green tea catechins, hinokitiol, lactoferrin, lactoperoxidase, or the like to enhance the effectiveness of the halitosis suppressant. The antimicrobial ingredients may be included in an amount of 0.01% or more and 10% or less, preferably 0.05% or more and 7% or less, and more preferably 0.1% or more and 5% or less, based on the raw materials of the functional layer.

As used herein, the term “saliva secretion-promoting agent” refers to a component that stimulates the salivary glands to promote saliva secretion. It may include saccharides, sweeteners, ascorbic acid, malic acid, and citric acid, and preferably include xylitol. The saliva secretion-promoting agent may be included in an amount of 30% or more and 95% or less, preferably 40% or more and 90% or less, and more preferably 50% or more and 85% or less, based on the raw materials of the functional layer.

A “lubricant” may be added to facilitate the compression and tablet ejection during manufacturing. Raw materials of the lubricant may include calcium stearate, magnesium stearate, stearic acid, sodium stearyl fumarate, or talc. The lubricant may be included in an amount of 0.1% or more and 10% or less, preferably 0.3% or more and 7% or less, and more preferably 0.5% or more and 5% or less, based on the raw materials of the functional layer.

The functional layer may include one or more binders selected from the group consisting of gelatin, alginate, pectin, starch, hydroxy methyl cellulose, hydroxy ethyl cellulose, hydroxypropyl methylcellulose, and polyvinyl acid. The binder may be included in an amount of 0.1% or more and 20% or less, preferably 3% or more and 17% or less, and more preferably 5% or more and 15% or less, based on the raw materials of the functional layer.

In addition, the functional layer or the adhesive layer may further include sweeteners such as stevia or saccharin to enhance taste, and flavoring agents such as peppermint oil or menthol to enhance aroma.

According to one embodiment of the present invention, the oral preparation may have a strength of 1000 gf or more and less than 4000 gf. If the strength is less than 1000 gf, there is a risk of breakage during manufacturing and transportation. Conversely, if it exceeds 4000 gf, it may be difficult to consume.

According to one embodiment of the present invention, the oral preparation may include an additional layer that performs one or more functions selected from the group consisting of surface coating, dissolution rate control, strength control, tooth surface polishing, whitening, and pH adjustment. Additionally, the layer performing a function may be composed of multiple layers.

### Experimental Example 1. Comparison of Adhesive Strength and Strength of Oral Preparations Based on Adhesive Polymers

### Preparation Example 1. Preparation of Comparative Examples 1 to 4 and Examples 1 to 3

To prepare adhesive layers for strength measurement, the same common ingredients were used, including excipients, pH adjusters, binders, and lubricants. Calcium carbonate (CaCO₃) was used as an excipient, sodium bicarbonate (NaHCO₃) as a pH adjuster, hydroxypropyl methylcellulose (HPMC) as a binder, and magnesium stearate as a lubricant.

Tablets were prepared by adding the respective raw materials for each preparation to a powder mixer and mixing for at least 30 minutes. Then, 300 mg of the mixture was placed into a 12 mm-diameter circular mold and compressed at a pressure of 5 mPa using a hydraulic press (Qmesys, QM900A) to manufacture tablets.

### Experimental Example 1-1. Adhesive Strength Evaluation of Oral Preparations Based on Adhesive Polymers

FIG. 3 is an image of a test process for measuring the adhesive strength of an oral preparation according to an embodiment of the present invention.

The adhesive strength evaluation was conducted under both normal conditions and high-saliva conditions. For measurements under normal conditions, 0.5 g of moisture that was added to prevent the gel from drying was removed before the measurement using a micropipette. In contrast, under high-saliva conditions, measurements were taken without removing the 0.5 g of moisture.

The adhesive strength of the prepared oral preparation samples was measured with reference to the literature (Annika Borde et al., Journal of Colloid and Interface Science 341 (2010) 255-260). The measurement was conducted using an oral mucosa-mimicking material to determine the force required for the oral preparation to detach from the material.

First, a 1% agarose gel was selected as the oral mucosa-mimicking material. Agarose at a concentration of 1% was dissolved in distilled water at a high temperature, and 10 g of the prepared gel solution was poured into a 50 mm Petri dish and allowed to solidify at room temperature. Then, 0.5 g of moisture was added to prevent the surface of the gel from drying, and the Petridish was stored.

The adhesive strength was measured by using a texture analyzer (TA.XT plus, TA Instruments) and a 35 mm aluminum probe. The tablet was fixed to the probe tip using double-sided tape, then pressed onto the surface of the 1% agarose gel, fixed to the ground, with a force of 50 gf for 30 seconds. The force required for detachment was then divided by the surface area of the tablet to calculate the adhesive strength of the sample in units of g/mm². To improve the accuracy of the measurements, five samples were prepared for each Preparation Example, and the average adhesive strength of the measured values was recorded for each Preparation Example.

### Experimental Example 1-2. Strength Evaluation of Oral Preparations Based on Adhesive Polymers

FIG. 4 is an image of a test process for measuring the strength of an oral preparation according to an embodiment of the present invention.

The strength of the prepared oral preparations was measured using the Crush test method. An oral preparation was placed vertically on the ground, with its circular face perpendicular to the surface. The strength of the preparation was then determined using a texture analyzer (TA.XT plus, TA Instruments) and a 35 mm aluminum probe. The applied force (gf) was measured over time, and the maximum force recorded at the point of fracture was used to determine the strength. To improve the accuracy of the measurements, five samples were prepared for each preparation, and the average strength of the measured values was recorded for each preparation.

TABLE 1

| | Comparative | Comparative | Comparative | Comparative | | | |
|---|---|---|---|---|---|---|---|
| | Examples 1 | Examples 2 | Examples 3 | Examples 4 | Examples 1 | Examples 2 | Examples 3 |
| CaCO₃ | 32 | 32 | 32 | 32 | 32 | 32 | 32 |
| NaHCO₃ | 5 | 5 | 5 | 5 | 5 | 5 | 5 |
| HPMC | 10 | 10 | 10 | 10 | 10 | 10 | 10 |
| Mg Stearate | 3 | 3 | 3 | 3 | 3 | 3 | 3 |
| CMC | 50 | 0 | 0 | 0 | 0 | 0 | 0 |
| PVP | 0 | 50 | 0 | 0 | 0 | 0 | 0 |
| Carbopol | 0 | 0 | 50 | 0 | 0 | 0 | 0 |
| (Polyacrylic acid) | | | | | | | |
| Gum arabic | 0 | 0 | 0 | 50 | 0 | 0 | 0 |
| Chicory dietary | 0 | 0 | 0 | 0 | 50 | 0 | 25 |
| fiber | | | | | | | |
| Cassava starch | 0 | 0 | 0 | 0 | 0 | 50 | 25 |
| Tablet | 71.3 | 97.0 | 210.5 | 130.2 | 1978.1 | 1444.6 | 1244.4 |
| strength(gf) | | | | | | | |
| Tablet adhesive | 0.318 | 0.097 | 0.301 | 0.269 | 0.352 | 0.387 | 0.325 |
| strength in | | | | | | | |
| normal | | | | | | | |
| condition(gf/mm²) | | | | | | | |
| Tablet adhesive | 0.244 | 0.143 | 0.202 | 0.241 | 0.302 | 0.323 | 0.274 |
| strength in high- | | | | | | | |
| saliva | | | | | | | |
| condition(gf/mm²) | | | | | | | |

As shown in Table 1, the experimental results show that the formulations of Examples 1 to 3 exhibited higher strength and adhesive strength compared to those of Comparative Examples, with less reduction in adhesive strength under high-saliva conditions.

### Experimental Example 2. Comparison of Strength and Adhesive Strength of Oral Preparations Based on Excipients

### Preparation Example 2. Preparation of Comparative Examples 2, 5, and 6, and Examples 4 to 7

To prepare adhesive layers for strength measurement, the same common ingredients were used, including pH adjusters, binders, lubricants, and adhesive polymers. Sodium bicarbonate (NaHCO₃) was used as a pH adjuster, hydroxypropyl methylcellulose (HPMC) as a binder, magnesium stearate as an activator, and carboxymethyl cellulose (CMC) as an adhesive polymer.

Oral preparations were prepared by adding the respective raw materials for each Preparation Example to a powder mixer and mixing for at least 30 minutes. Then, 300 mg of the mixture was placed into a 12 mm-diameter circular mold and compressed at a pressure of 5 mPa using a hydraulic press (Qmesys, QM900A) to manufacture tablets.

The prepared oral preparations were stored in containers containing drying silica without any additional drying process. Subsequently, strength and adhesive strength were measured for each preparation.

The experiment was conducted by measuring the strength and adhesive strength of oral preparations formulated with different excipients.

TABLE 2

| | Comparative | Comparative | Comparative | | | | |
|---|---|---|---|---|---|---|---|
| | Examples 1 | Examples 5 | Examples 6 | Examples 4 | Examples 5 | Examples 6 | Examples 7 |
| CaCO₃ | 32 | 0 | 0 | 0 | 0 | 0 | 0 |
| Zeolite | 0 | 32 | 0 | 0 | 0 | 0 | 0 |
| 50 μm silica | 0 | 0 | 32 | 0 | 0 | 0 | 0 |
| 7 nm silica | 0 | 0 | 0 | 32 | 0 | 0 | 0 |
| 12 nm silica | 0 | 0 | 0 | 0 | 32 | 0 | 0 |
| 15 μm silica | 0 | 0 | 0 | 0 | 0 | 32 | 0 |
| 25 μm silica | 0 | 0 | 0 | 0 | 0 | 0 | 32 |
| NaHCO₃ | 5 | 5 | 5 | 5 | 5 | 5 | 5 |
| HPMC | 10 | 10 | 10 | 10 | 10 | 10 | 10 |
| Mg Stearate | 3 | 3 | 3 | 3 | 3 | 3 | 3 |
| CMC | 50 | 50 | 50 | 50 | 50 | 50 | 50 |
| Tablet | 71.3 | 20.2 | 89.7 | 3093.7 | 2295.9 | 2147.6 | 1489.8 |
| strength(gf) | | | | | | | |
| Tablet adhesive | 0.318 | 0.291 | 0.289 | 0.329 | 0.378 | 0.368 | 0.487 |
| strength in | | | | | | | |
| normal | | | | | | | |
| condition(gf/mm²) | | | | | | | |
| Tablet adhesive | 0.244 | 0.273 | 0.265 | 0.408 | 0.414 | 0.444 | 0.585 |
| strength in high- | | | | | | | |
| saliva | | | | | | | |
| condition(gf/mm²) | | | | | | | |

As shown in Table 2, the experimental results show that the formulations of Examples 4 to 7 exhibited higher strength and adhesive strength under both normal and high-saliva conditions compared to those of Comparative Examples 1 and 5. In particular, when the silica particle size was 50 μm or more, as in the preparation of Comparative Example 6, the excipient did not exhibit any effect in strength or adhesive strength enhancement.

### Experimental Example 3. Comparison of Strength and Adhesive Strength of Oral Preparations with Chicory Dietary Fiber or Cassava Starch from which Linamarin has been Removed as an Adhesive Polymer, and Silica as a Binder

### Preparation Example 3. Preparation of Examples 8 and 9

To prepare adhesive layers for strength measurement, the same common ingredients were used, including pH adjusters, binders, and lubricants. Sodium bicarbonate (NaHCO₃) was used as a pH adjuster, hydroxypropyl methylcellulose (HPMC) as a binder, and magnesium stearate as a lubricant.

The oral preparations were prepared by adding the respective raw materials for each Preparation Example to a powder mixer and mixing for at least 30 minutes. Then, 300 mg of the mixture was placed into a 12 mm-diameter circular mold and compressed at a pressure of 5 mPa using a hydraulic press (Qmesys, QM900A) to manufacture tablets.

The prepared oral preparations were stored in containers containing drying silica without any additional drying process. Subsequently, strength and adhesive strength measurements were conducted for each preparation sample.

The experiment was conducted in the same manner as in Experimental Example 1.

TABLE 3

| | Compar- | | |
|---|---|---|---|
| | ative | Exam- | Exam- |
| | Examples 1 | ples 8 | ples 9 |
| CaCO₃ | 32 | 0 | 0 |
| 25 μm silica | 0 | 32 | 32 |
| NaHCO₃ | 5 | 5 | 5 |
| HPMC | 10 | 10 | 10 |
| Mg Stearate | 3 | 3 | 3 |
| CMC | 50 | 0 | 0 |
| Chicory dietary | 0 | 50 | 0 |
| fiber | | | |
| Cassava starch | 0 | 0 | 50 |
| Tablet | 71.3 | 2844.3 | 1787.6 |
| strength(gf) | | | |
| Tablet adhesive | 0.318 | 0.403 | 0.431 |
| strength in | | | |
| normal | | | |
| condition | | | |
| (gf/mm²) | | | |
| Tablet adhesive | 0.244 | 0.380 | 0.470 |
| strength in | | | |
| high-saliva | | | |
| condition | | | |
| (gf/mm²) | | | |

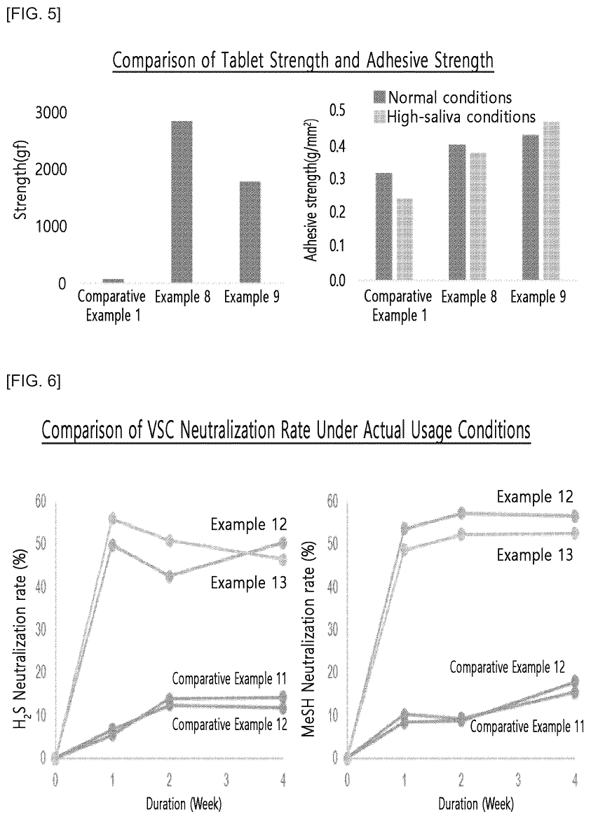

FIG. 5 is a graph comparing the adhesive strength of oral preparations according to an embodiment of the present invention. As shown in FIG. 5 and Table 3, the experimental results indicate that the formulations of Examples 8 and 9 exhibit higher strength and adhesive strength compared to that of Comparative Example 1. Furthermore, when compared to Examples 1 and 2, the replacement of the excipient with silica resulted in enhancement of both the strength and adhesive strength.

### Experimental Example 4. Comparison of Oral Mucosal Irritation Based on Adhesive Polymers

### Preparation Example 4. Preparation of Comparative Examples 7 to 10, and Examples 10 and 11

For comparison according to the adhesive polymer, the Comparative Examples were composed of carboxymethyl cellulose (CMC), polyvinylpyrrolidone (PVP), Carbopol (polyacrylic acid), and gum arabic, while the Examples were composed of chicory dietary fiber or cassava starch.

In this experiment, the degree of irritation was compared by evaluating cytotoxicity to assess oral mucosal irritation. The cytotoxicity evaluation was conducted by culturing V79-4 cells (Chinese Hamster, CCL of lung tissue fibroblasts) for 24 hours, followed by diluting each adhesive polymer to an appropriate concentration (arbitrarily selected between 1% and 10%) using a dilutable solvent such as medium (3% FBS), DPBS, or DMSO. Subsequently, the medium was treated with either the undiluted solution or a 1/100 dilution, followed by a 24-hour incubation. After incubation, MTT reagent was used to measure the absorbance, thereby determining cell viability. The results were used to determine the half-maximal inhibitory concentration (IC₅₀), which refers to the concentration at which 50% of cells undergo cell death, and Cellular Toxicity index (CTI).

TABLE 4

| | Comparative | Comparative | Comparative | Comparative | | |
|---|---|---|---|---|---|---|
| | Examples 7 | Examples 8 | Examples 9 | Examples 10 | Examples 10 | Examples 11 |
| adhesive | CMC | PVP | Carbopol | Gum | Chicory | Cassava |
| polymer | | | (Polyacrylic | arabic | dietary | starch |
| | | | acid) | | fiber | |
| IC50 | 0.098 | 2.945 | 0.652 | 1.336 | 8.405 | 7.940 |
| CTI | Moderate | Extremely | Low | Extremely | Extremely | Extremely |
| | | Low | | Low | Low | Low |

As shown in Table 4, the IC₅₀ values of Examples 10 and 11 are relatively higher compared to those of Comparative Examples 7 to 10, indicating lower toxicity. Thus, the CTI was found to be extremely low.

### Experimental Example 5. Comparison of Halitosis Improvement Effect of Oral Preparations Based on Addition of Zinc Ions (Zn²⁺) and Copper Ions (Cu²⁺)

### Preparation Example 5. Preparation of Comparative Examples 11 and 12, and Examples 12 and 13

The oral preparations for halitosis improvement evaluation were prepared as shown in Table 5, using the same common raw materials including excipients, flavor enhancers, pH adjusters, binders, and lubricants. As excipients, xylitol was used in the upper layer, while calcium carbonate (CaCO₃) was used in the lower layer. The upper layer contained sodium saccharin, L-menthol, and peppermint extract powder as flavor enhancers, while the lower layer contained sodium bicarbonate (NaHCO₃) as a pH adjuster. Hydroxypropyl methylcellulose (HPMC) was used as a binder, and magnesium stearate was used as a lubricant.

TABLE 5

| | | Compar- | Compar- | | |
|---|---|---|---|---|---|
| | raw | ative | ative | Exam- | Exam- |
| division | material | Examples 11 | Examples 12 | ples 12 | ples 13 |
| functional | Xylitol | 85.85 | 85.85 | 82.34 | 82.34 |
| layer | Zn | 0.00 | 0.00 | 2.32 | 2.32 |
| | Gluconate | | | | |
| | Cu | 0.00 | 0.00 | 1.19 | 1.19 |
| | Gluconate | | | | |
| | Na | 0.50 | 0.50 | 0.50 | 0.50 |
| | Saccharin | | | | |
| | L-menthol | 0.50 | 0.50 | 0.50 | 0.50 |
| | HPMC | 10.00 | 10.00 | 10.00 | 10.00 |
| | Mg | 3.00 | 3.00 | 3.00 | 3.00 |
| | stearate | | | | |
| | Peppermint | 0.15 | 0.15 | 0.15 | 0.15 |
| | extract | | | | |
| | powder | | | | |
| | Total | 100.00 | 100.00 | 100.00 | 100.00 |
| adhesive | CaCO₃ | 32 | 32 | 32 | 32 |
| layer | NaHCO₃ | 5 | 5 | 5 | 5 |
| | HPMC | 10 | 10 | 10 | 10 |
| | Mg | 3 | 3 | 3 | 3 |
| | Searate | | | | |
| | Chicory | 50 | 0 | 50 | 0 |
| | dietary | | | | |
| | fiber | | | | |
| | Cassava | 0 | 50 | 0 | 50 |
| | starch | | | | |
| | Total | 100.00 | 100.00 | 100.00 | 100.00 |

The oral tablets were prepared by adding the raw materials of the corresponding layer for each preparation to a powder mixer according to the ratio indicated in the table and mixing for at least 30 minutes. Then, 50 mg of the mixture for the lower layer was placed into a 12 mm-diameter circular mold and flattened. Subsequently, 300 mg of the mixture for the upper layer was injected into the mold, followed by compression at a pressure of 5 mPa using a hydraulic press (Qmesys, QM900A).

The prepared oral preparations were stored in containers containing drying silica without any additional drying process.

### Experimental Example 5-1. Comparison of VSC-Neutralizing Effect of Oral Preparations Based on Addition of Zinc Ions (Zn²⁺) and Copper Ions (Cu²⁺)

In this experiment, oral preparations for improving halitosis were prepared and evaluated for VSC-neutralizing effect. A 100 μL of a malodor source sample (containing approximately 1250 ppb of H₂S and 1000 ppb of MeSH) was placed into a sealed 10 mL headspace vial. Then, the tablets from the Preparation Examples were each pulverized and dissolved in purified water to prepare a 25% tablet suspension, of which 1 g was added to the vial. Then, after allowing the mixture to stand for 3 minutes, the concentrations of H₂S and MeSH were measured using an OralChroma (Abimedical, Abilit Corp.).

TABLE 6

| | Compar- | Compar- | | |
|---|---|---|---|---|
| neutralization | ative | ative | Exam- | Exam- |
| rate(%) | Examples 11 | Examples 12 | ples 12 | ples 13 |
| H₂S | 90.13 | 85.41 | 99.64 | 99.15 |
| MeSH | 15.42 | 19.72 | 93.52 | 96.43 |

FIG. 6 is a graph comparing the VSC neutralization rate of oral preparations with or without a halitosis suppressant, according to one embodiment of the present invention. As shown in FIG. 6 and Table 6, the preparations of Examples 12 and 13 exhibit a superior halitosis improvement effect compared to those of Comparative Examples 11 and 12.

### Experimental Example 5-2. Comparison of VSC Neutralization Rates of Oral Preparations Based on Addition of Zinc Ions (Zn2+) and Copper Ions (Cu2+) Under Actual Usage Conditions

An evaluation of the halitosis improvement effect of oral preparations was performed by preparing the oral preparations and measuring the VSC neutralization rate under actual usage conditions. Twenty-eight participants, randomly divided into four groups, were provided with different samples and instructed to consume the oral preparation twice a day. The participants consumed the oral preparations for halitosis improvement for four weeks. The VSC neutralization rate was measured each week, and after four weeks, the rate of reduction of four types of halitosis-causing bacteria were measured to evaluate the halitosis improvement effect.

The concentrations of H₂S and MeSH were measured at week 0 using OralChroma (Abimedical, Abilit Corp.). The concentrations of H₂S and MeSH were then measured each week, and the neutralization rate was calculated using the following equation.

$$VSC neutralization rate \% = ( ( concentration of VSC measured each week - initial concentration of VSC ) / ( initial concentration of VSC ) ) \times 100$$

TABLE 7

| | | Compar- | Compar- | | |
|---|---|---|---|---|---|
| Halitosis | | ative | ative | Exam- | Exam- |
| ingredients | period | Examples 11 | Examples 12 | ples 12 | ples 13 |
| H₂S | After 1 | 5.4 | 6.7 | 49.9 | 56.1 |
| | week | | | | |
| | After 2 | 13.9 | 12.4 | 42.6 | 51 |
| | weeks | | | | |
| | After 4 | 14.3 | 11.7 | 50.4 | 46.5 |
| | weeks | | | | |
| MeSH | After 1 | 10.2 | 8.4 | 53.7 | 48.7 |
| | week | | | | |
| | After 2 | 9.3 | 8.8 | 57.4 | 52.4 |
| | weeks | | | | |
| | After 4 | 15.4 | 17.9 | 56.7 | 52.7 |
| | weeks | | | | |

As shown in Table 7, the preparations of Examples 12 and 13 exhibit a higher VSC neutralization rate under actual usage conditions compared to those of Comparative Examples 11 and 12. Therefore, it was confirmed that the preparations of Examples 12 and 13 exhibited superior halitosis improvement effect compared to those of Comparative Examples 11 and 12.

Experimental Example 5-3. Comparison of Reduction Rate of Halitosis-Causing Bacteria in Oral Preparations Based on Addition of Zinc Ions (Zn²⁺) and Copper Ions (Cu²*) Under Actual Usage Conditions

The halitosis improvement effect of oral was evaluated by preparing the oral preparations and measuring the rate of reduction of halitosis-causing bacteria under actual usage conditions. The experiment was conducted by measuring the rate of reduction of halitosis-causing bacteria using the following method. At the beginning of the experiment, participants gargled with a mouthwash solution (LG Household & Health Care Easyperio) to quantitatively evaluate the oral bacterial strains. The collected mouthwash samples were analyzed using PCR to quantify the oral microorganisms of the participants. After consuming the oral preparations for four weeks, the participants gargled again with a mouthwash solution (LG Household & Health Care Easyperio) for one minute, and collected mouthwash samples were analyzed using PCR to quantify the oral microorganisms of the participants. Then, the rate of reduction of halitosis-causing bacteria was obtained using the following equation.

$$rate of reduction of halitosis - causing bacteria \% = ( ( number of halitosis - causing bacteria in week 4 - initial number of halitosis - causing bacteria ) / ( initial number of halitosis - causing bacteria ) ) \times 100$$

TABLE 8

| Halitosis- | Compar- | Compar- | | |
|---|---|---|---|---|
| causing | ative | ative | Exam- | Exam- |
| bacteria | Examples 11 | Examples 12 | ples 12 | ples 13 |
| F. nucleatum | 7.8 | 10.8 | 28.7 | 40.4 |
| P. intermedia | 9.3 | 10.1 | 57.3 | 48.1 |
| P. nigrescens | 20.4 | 17.9 | 80.5 | 75.4 |
| A. viscosus | 6.2 | 5.9 | 15.1 | 10.7 |

As shown in Table 8, Examples 12 and 13 exhibit a higher reduction rate of the four halitosis-causing bacteria under actual usage conditions compared to Comparative Examples 11 and 12. Therefore, it was confirmed that Examples 12 and 13 exhibited a higher halitosis improvement effect compared to Comparative Examples 11 and 12.

### Experimental Example 6. Evaluation on User Satisfaction and Oral Irritation of Oral Preparations Based on Adhesive Polymers

### Preparation Example 6. Preparation of Comparative Examples 13 to 16, and Examples 12 and 13

To prepare oral preparations for sensory evaluation, the same common ingredients were used, including excipients, flavor enhancers, pH adjusters, binders, and lubricants. As excipients, xylitol was used in the upper layer, while calcium carbonate (CaCO₃) was used in the lower layer. The upper layer contained sodium saccharin, L-menthol, and peppermint extract powder as flavor enhancers, and the lower layer contained sodium bicarbonate (NaHCO₃) as a pH adjuster. Hydroxypropyl methylcellulose (HPMC) was used as a binder, and magnesium stearate was used as a lubricant.

The oral tablets were prepared by adding the raw materials of the corresponding layer for each Preparation Example to a powder mixer according to the ratio indicated in the table and mixing for at least 30 minutes. Then, 50 mg of the mixed raw materials of the lower layer was placed into a 12 mm-diameter circular mold and flattened. Subsequently, 300 mg of the raw materials of the upper layer was injected into the mold, followed by compression at a pressure of 5 MPa using a hydraulic press (Qmesys, QM900A).

The prepared oral preparations were stored in a container containing drying silica without any additional drying process.

The experiment was conducted by preparing oral preparations for improving halitosis with different adhesive polymers and evaluating user satisfaction and oral irritation. Thirty participants, randomly divided into six groups, were provided with different types of samples and instructed to consume the oral preparation twice a day. After one week of use, a sensory evaluation was conducted on aroma, taste, irritation, and freshness. The evaluation was conducted using a scoring system ranging from a minimum of 0 to a maximum of 5 points. The average value of the response from the participants was calculated to determine the score for each category.

TABLE 9

| | | Comparative | Comparative | Comparative | Comparative | | |
|---|---|---|---|---|---|---|---|
| division | raw material | Examples 13 | Examples 14 | Examples 15 | Examples 16 | Examples 12 | Examples 13 |
| functional | Xylitol | 82.34 | 82.34 | 82.34 | 82.34 | 82.34 | 82.34 |
| layer | Zn Gluconate | 2.32 | 2.32 | 2.32 | 2.32 | 2.32 | 2.32 |
| | Cu Gluconate | 1.19 | 1.19 | 1.19 | 1.19 | 1.19 | 1.19 |
| | Na Saccharin | 0.50 | 0.50 | 0.50 | 0.50 | 0.50 | 0.50 |
| | L-menthol | 0.50 | 0.50 | 0.50 | 0.50 | 0.50 | 0.50 |
| | HPMC | 10.00 | 10.00 | 10.00 | 10.00 | 10.00 | 10.00 |
| | Mg stearate | 3.00 | 3.00 | 3.00 | 3.00 | 3.00 | 3.00 |
| | Peppermint | 0.15 | 0.15 | 0.15 | 0.15 | 0.15 | 0.15 |
| | extract | | | | | | |
| | powder | | | | | | |
| | Total | 100.00 | 100.00 | 100.00 | 100.00 | 100.00 | 100.00 |
| adhesive | CaCO₃ | 32 | 32 | 32 | 32 | 32 | 32 |
| layer | NaHCO₃ | 5 | 5 | 5 | 5 | 5 | 5 |
| | HPMC | 10 | 10 | 10 | 10 | 10 | 10 |
| | Mg Searate | 3 | 3 | 3 | 3 | 3 | 3 |
| | CMC | 50 | 0 | 0 | 0 | 0 | 0 |
| | PVP | 0 | 50 | 0 | 0 | 0 | 0 |
| | Carbopol | 0 | 0 | 50 | 0 | 0 | 0 |
| | Gum arabic | 0 | 0 | 0 | 50 | 0 | 0 |
| | Chicory | 0 | 0 | 0 | 0 | 50 | 0 |
| | dietary fiber | | | | | | |
| | Cassava | 0 | 0 | 0 | 0 | 0 | 50 |
| | starch | | | | | | |
| | Total | 100.00 | 100.00 | 100.00 | 100.00 | 100.00 | 100.00 |
| Sensory | Aroma | 3.62 | 3.6 | 3.41 | 3.55 | 3.86 | 3.75 |
| evaluation | Taste | 3.25 | 3.26 | 3.14 | 3.4 | 3.43 | 3.88 |
| | Low irritation | 2.45 | 3.43 | 1.17 | 3.11 | 4 | 4.02 |
| | Freshness | 2.98 | 3 | 3.05 | 3.1 | 3.14 | 3.27 |

As shown in Table 9, the sensory evaluation results indicate that the preparations of Comparative Examples 13 to 16 and Examples 12 to 13 received similar scores in aroma, taste, and freshness. However, for low irritation, the preparations of Examples 12 and 13 received higher scores than those of Comparative Examples 13 to 16, indicating that the preparations of Examples 12 and 13 caused less irritation compared to those of Comparative Examples 13 to 16.

### Experimental Example 7. Measurement of Dissolution Rate of Oral Preparations Based on Adhesive Polymers

### Preparation Example 7. Preparation of Examples 14 and 15, and Comparative Example 17

As an oral preparation for measuring the dissolution rate, Comparative Example 17 was prepared using the mucoadhesive product (Oracoat Xylimelts, product usage duration (dissolution rate): 1 to 4 hours during the day and 4 to 8 hours at night) after attachment to the oral mucosa. Additionally, to match the tablet weight of the preparation of Comparative Example 17 (approximately 700 mg), Examples 14 and 15 had the same compositions as those of Example 12 and 13, respectively, but used 100 mg of the raw materials for the adhesive layer and 600 mg of the raw materials for the functional layer for tableting. To prepare the oral preparations as shown in Table 9, the same common ingredients were used, including excipients, flavor enhancers, pH adjusters, binders, and lubricants. As excipients, xylitol was used in the upper layer, while calcium carbonate (CaCO₃) was used in the lower layer. The upper layer contained sodium saccharin, L-menthol, and peppermint extract powder as flavor enhancers, and the lower layer contained sodium bicarbonate (NaHCO₃) as a pH adjuster. Hydroxypropyl methylcellulose (HPMC) was used as a binder, and magnesium stearate was used as a lubricant.

The oral tablets were prepared by adding the raw materials of the corresponding layer for each Preparation Example to a powder mixer according to the ratio indicated in Table 9 and mixing for at least 30 minutes. Then, 100 mg of the mixed raw materials of the adhesive layer was placed into a 12 mm-diameter circular mold and flattened. Subsequently, 600 mg of the raw materials of the upper layer was injected into the mold, followed by compression at a pressure of 5 MPa using a hydraulic press (Qmesys, QM900A).

The prepared oral preparations were stored in a container containing drying silica without any additional drying process.

The experiment was conducted by preparing oral preparations for improving halitosis with different adhesive polymers and measuring the dissolution rate. Tablets of the preparations of Example 14, Example 15, and Comparative Example 17 were each placed in a tea bag, and the dissolution rate in 100 g of distilled water at 37° C. was measured. For each preparation, the tea bag was removed from the distilled water after 1 hour, 2 hours, and 4 hours. The removed tea bags were then dried in a 60° C. oven for 24 hours, and the weight of the tea bag as measured. The dissolution rate was calculated using the following equation.

$$Dissolution rate \% = ( ( weight before experiment - weight per hour after drying ) / ( weight before experiment ) ) \times 100$$

TABLE 10

| | | | Compar- |
|---|---|---|---|
| | Exam- | Exam- | ative |
| Time(h) | ples 14 | ples 15 | Examples 17 |
| 0 | 0 | 0 | 0 |
| 1 | 48.6 | 43.8 | 38.3 |
| 2 | 68.7 | 65.2 | 60.3 |
| 3 | 90.1 | 87.6 | 94.8 |

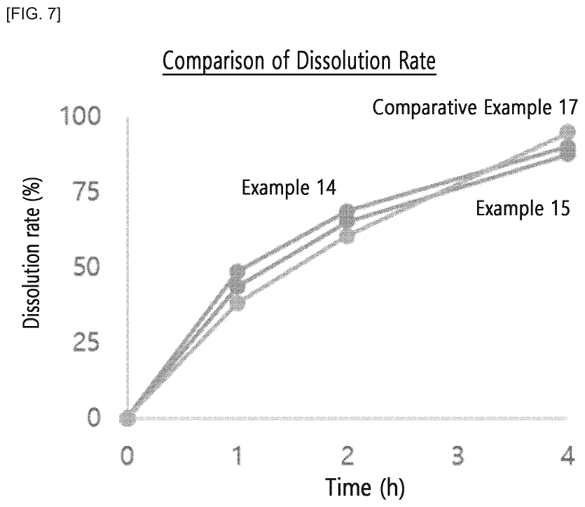

FIG. 7 is a graph comparing the dissolution rate of oral preparations according to an embodiment of the present invention. As shown in Table 10 and FIG. 7, the dissolution rate evaluation results indicate that the preparations of Examples 14 and 15 exhibit a similar dissolution rate compared to that of Comparative Example 17. However, the preparations of Examples 14 and 15 were found to disintegrate slightly more slowly than that of Comparative Example 17, suggesting that the active ingredients were released in a more sustained manner, thereby demonstrating a superior sustained-release effect.

The present invention has been described with reference to preferred embodiments; however, a person of ordinary skill or knowledge in the art would understand that various modifications and changes may be made without departing from the spirit and scope of the invention as defined in the following claims.

Therefore, the technical scope of the present invention should not be limited to the detailed description provided in the specification but should be defined by the claims.

1. An oral preparation, comprising:
an adhesive layer comprising an adhesive polymer; and a functional layer,
wherein the adhesive polymer comprises one or more selected from the group consisting of cassava starch in which linamarin is removed and chicory dietary fiber.

2. The oral preparation of claim 1,
wherein the functional layer provides one or more functions selected from suppressing halitosis, promoting saliva secretion, reducing tooth plaque, reducing gingivitis, reducing periodontitis, alleviating periodontal disease, and alleviating dental caries.

3. The oral preparation of claim 1,
wherein the oral preparation is in the form of a bilayer tablet.

4. The oral preparation of claim 1,
wherein the adhesive layer further comprises silica as an excipient,
and wherein the oral preparation is in any one formulation selected from the group consisting of halitosis removing agents, dry mouth relievers, solid oral cleansers, solid toothpastes, and sustained-release candies.

5. The oral preparation of claim 4,
wherein the excipient is included in an amount of 20 wt % to 40 wt % based on the total weight of the raw materials of the adhesive layer.

6. The oral preparation of claim 4,
wherein the silica has a particle diameter of 1 nm or more and 50 μm or less.

7. The oral preparation of claim 1,
wherein the functional layer comprises, as a halitosis suppressant, one or more selected from the group consisting of salts of copper ions (Cu²⁺), zinc ions (Zn²⁺), and tin ions (Sn²⁺).

8. The oral preparation of claim 7,
wherein the functional layer comprises the halitosis suppressant in an amount of 0.1 wt % to 5 wt % based on the total weight of the functional layer.

9. The oral preparation of claim 1,
wherein the functional layer comprises a saliva stimulant in an amount of 50 wt % to 80 wt % based on the total weight of the functional layer.

10. The oral preparation of claim 1,
wherein the adhesive layer is included in an amount of 2 wt % to 50 wt % based on the total weight of the oral preparation.

11. The oral preparation of claim 1,
wherein the functional layer is included in an amount of 20 wt % to 85 wt % based on the total weight of the oral preparation.

12. The oral preparation of claim 1,
wherein the oral preparation exhibits a strength of 1000 gf or more and less than 4000 gf.

13. The oral preparation of claim 1,
wherein the oral preparation further comprises a layer that performs one or more functions selected from the group consisting of surface coating, dissolution rate control, strength control, tooth surface polishing, whitening, and pH adjustment.

14. An oral adhesive composition comprising a polymer, wherein the polymer comprises one or more selected from the group consisting of cassava starch in which linamarin is removed and chicory dietary fiber and functions as an oral adhesive.
